(19) United States
(12) Patent Application Publication
(10) Pub. No.: US 2013/0253057 A1
(43) Pub. Date: Sep. 26, 2013

(54) Personal Care Compositions and Methods

(71) Applicant: THE PROCTER & GAMBLE COMPANY, Cincinnati, OH (US)

(72) Inventors: Karl Shiqing WEI, Mason, OH (US); Qing STELLA, Cincinnati, OH (US); Wei JI, Cincinnati, OH (US); Gerald John GUSKEY, Cincinnati, OH (US); Stevan David JONES, Singapore (SG); Kunal Virendra GUJRATY, Bangalore (IN); James Terence WESCOTT, Portishead (GB)

(73) Assignee: The Procter & Gamble Company, Cincinnati, OH (US)

(21) Appl. No.: 13/848,271

(22) Filed: Mar. 21, 2013

Related U.S. Application Data
(60) Provisional application No. 61/614,119, filed on Mar. 22, 2012.
(60) Provisional application No. 61/647,944, filed on May 16, 2012.

(51) Int. Cl. A61K 8/92; A61Q 19/00

(52) U.S. Cl. CPC A61K 8/92; A61Q 19/007
USPC 514/549; 514/552

(57) ABSTRACT

A personal care composition includes a cleansing phase and a benefit phase, the cleansing phase having a surfactant and the benefit phase having a lipid bilayer structurant. Methods to enhance skin hydration are also provided.

### TECHNICAL FIELD

The present disclosure generally relates to a personal care composition comprising a lipid bilayer structurant and methods corresponding thereto.

### BACKGROUND

Cleansing the skin is an activity that has been done for millennia. Skin cleansing and methods therefore have involved the utilization of soaps, body washes, and other personal cleansing compositions. Personal cleansing compositions can be formulated to deliver benefit agents while maintaining physical integrity of the compositions. The ability to deposit benefit agents and hydrate the skin while maintaining the physical integrity can be an important benefit for such compositions. Oils, for example, are a type of benefit agent for skin hydration enhancement. However, it is known that many such benefit agents can exhibit poor deposition characteristics or even poor hydration benefits even with sufficient deposition. Achieving a proper balance between stability in a composition and performance properties such as enhanced skin hydration can be a difficult task, and as such, it is desirable to provide a personal care composition to effectively enhance skin hydration.

### SUMMARY

A method of enhancing skin hydration, the method includes applying a personal cleansing composition to skin and rinsing the personal care composition from the skin. The personal cleansing composition can comprise a cleansing phase; and a benefit phase comprising a hydrophobic benefit agent and a lipid bilayer structurant. The skin exhibits a hydration improvement of about 1.2 Corneometer Units or more, as compared to a water control, at 24 hours after one application.

A method of enhancing skin hydration, the method comprising formulating a personal care composition and instructing an individual to apply the composition to skin. The personal care composition may comprise a cleansing phase; and a benefit phase comprising a hydrophobic benefit agent and a lipid bilayer structurant. The skin exhibits a hydration improvement of about 1.2 Corneometer Units or more, as compared to a water control, at 24 hours after one application.

A method of improving skin hydration, the method comprising providing a personal care composition and applying the personal care composition to the skin of an individual. The personal care composition comprising a cleansing phase; and an anhydrous benefit phase comprising a hydrophobic benefit agent and from about 0.05% to about 5% of a lipid bilayer structurant comprising glyceryl monooleate, glyceryl monostearate, glyceryl monolaurate, or a combination thereof. The skin exhibits a hydration improvement of about 1.2 Corneometer Units or more, as compared to a water control, at 24 hours after one application.

### BRIEF DESCRIPTION OF THE DRAWINGS

FIG. 1 is an image of a lipid bilayer in both the cis and trans configurations;

FIG. 2 is a graph showing change in dry skin grade over a treatment period; and

FIG. 3 is a graph showing change in transepidermal water loss over a treatment period.

### DETAILED DESCRIPTION

It is believed the scope of the present invention will be better understood from the following description.

The devices, apparatuses, methods, components, and/or compositions of the present invention can include, consist essentially of, or consist of, the components of the present invention as well as other ingredients described herein. As used herein, “consisting essentially of” means that the devices, apparatuses, methods, components, and/or compositions may include additional ingredients, but only if the additional ingredients do not materially alter the basic and novel characteristics of the claimed devices, apparatuses, methods, components, and/or compositions.

All percentages and ratios used herein are by weight of the total composition and all measurements made are at 25° C., unless otherwise designated.

All measurements used herein are in metric units unless otherwise specified.

### I. Definitions

As used herein, the following terms shall have the meaning specified thereafter: “Anhydrous” refers to those compositions, and components thereof, which are substantially free of water.

“Associative polymer” refers to a water-dispersible polymer comprising hydrophobic groups at an end or pendants to a hydrophilic backbone.

“Dry skin” refers to a term used by consumers, cosmetic scientists, and dermatologists. Dry skin can be characterized by a rough, scaly and/or flaky skin surface, especially in low humidity conditions and is often associated with the somatory sensations of tightness, itch, and/or pain.

“Lipid bilayer structurant” refers to molecules that embed themselves in the skin lipid bilayer to promote the ordering of the bilayers, resulting in improved barrier function and increased skin hydration.

“Multiphase” refers to compositions comprising at least two phases which can be chemically distinct (e.g., a cleansing phase and a benefit phase). Such phases can be in direct physical contact with one another. A personal care composition can be a multiphase personal care composition where phases of the personal care composition can be blended or mixed to a significant degree, but still be physically distinct. In these situations, the physical distinctiveness is undetectable to the naked eye. In another example, the personal care composition can be a multiphase personal care composition where phases of the personal care composition can be made to occupy separate but distinct physical spaces inside a package in which the phases can be stored. In such an example, the phases can be stored such that the phases are not in direct contact with one another (i.e., the phases are separated by a barrier). The personal care composition can also be a multiphase personal care composition where the phases are in physical contact and are visually distinct (i.e., the phases are not separated by a barrier and the phases are not emulsified or mixed to any significant degree). Visually distinct phases can take many forms (e.g., phases can appear as striped, marbled). The personal care composition can also include a combination of one or more of the above multiphase personal care compositions. For example, one blended multiphase personal care composition can be stacked with another blended multiphase personal care composition to form a striped configuration. Another example includes a blended multiphase personal care compositions distinguishable by color stacked as stripes wherein the blended multiphase personal care compositions can be otherwise similar in average composition.

“Non-associative polymer” refers to a water-dispersible polymer with a relatively uniform hydrophilic backbone lacking hydrophobic groups.

“Package” refers to any suitable container for a personal care composition including but not limited to a bottle, tottle, tube, jar, non-aerosol pump, and combinations thereof.

“Personal care composition” refers to compositions intended for topical application to skin and/or hair. Personal care compositions can be rinse-off formulations, in which the product can be applied topically to the skin and/or hair and then subsequently rinsed within seconds to minutes of application with water. The product could also be wiped off using a substrate. In either case, it is believed at least a portion of the product is left behind (i.e., deposited) on the skin. The personal care composition may also be a leave-on, see for example U.S. Pat. No. 5,833,998. The personal care compositions can also be used as shaving aids. The personal care compositions can be extrudable or dispensable from a package. The personal care compositions can be in the form of, for example, a liquid, semi-liquid cream, lotion, or gel. Examples of personal care compositions can include but are not limited to bar soap, shampoo, conditioning shampoo, body wash, moisturizing body wash, shower gels, skin cleansers, cleansing milks, hair and body wash, in shower body moisturizer, pet shampoo, shaving preparations, cleansing compositions used in conjunction with a disposable cleansing cloth, cosmetics, moisturizers, deodorant, antiperspirant, skin care compositions, etc.

“STnS” refers to sodium trideceth (n) sulfate, wherein n can define the average number of moles of ethoxylate per molecule.

“Stable” refers to a personal care composition having a viscosity change of about 30% or less from an initial viscosity value after being rapidly aged for 10 days at 50° C. as described in the T-Bar Viscosity Method below.

“Structured” refers to having a rheology that can confer stability on the personal care composition. A degree of structure can be determined by characteristics determined by a Zero Shear Viscosity Method described below. Accordingly, a structured cleansing phase of the personal care composition can be considered to be structured if the structured cleansing phase has a Zero Shear Viscosity of about 20 Pascal-seconds (Pa-s) or more, about 200 Pa-s or more, about 500 Pa-s or more, about 1,000 Pa-s or more, about 1,500 Pa-s or more, or about 2,000 Pa-s or more. Other methods for determining characteristics which can define a degree of structure are described in U.S. Patent Application Publication No. 2012/0009285.

The phrase “substantially free of” as used herein, unless otherwise specified means that the personal care composition comprises less than about 1%, or even less than about 0.1% of the stated ingredient. The term “free of” as used herein means that the personal care composition comprises 0% of the stated ingredient, that is, the ingredient has not been added to the personal care composition, however, these ingredients may incidentally form as a by-product or a reaction product of the other components of the personal care composition.

“Visually distinct” generally refers to a region of the multiphase personal care composition having one average composition, as distinct from another region having a different average composition, wherein the regions can be visible to the unaided naked eye. This would not preclude distinct regions from comprising two similar multiphase personal care compositions or phases where one multiphase personal care composition or phase can comprise certain pigments, dyes, particles, and various optional ingredients, hence providing a region of different average composition (e.g., different textures or different colors).

### II. Personal Care Compositions

There are many factors that contribute to the hydration level of the skin. These can include, for example, the skin's ability to retain its own hydration and whether there have been any products applied to the skin to increase the barrier properties of the skin. Hydration is important to the skin because drier skin is more easily damaged and can look older faster.

Simplistically, the skin contains 3 main layers which are the epidermis, the dermis, and the basement membrane. The main function of the epidermis is to act as the body's protective barrier, holding in vital water and keeping out pathogens. The epidermis itself is made of multiple layers, one of which is the stratum corneum. Within the stratum corneum is a lamellar lipid bilayer which plays an important role in maintaining the barrier properties of the skin. When the lipid bilayer is disrupted and becomes less organized, then its ability to function as a barrier is negatively impacted.

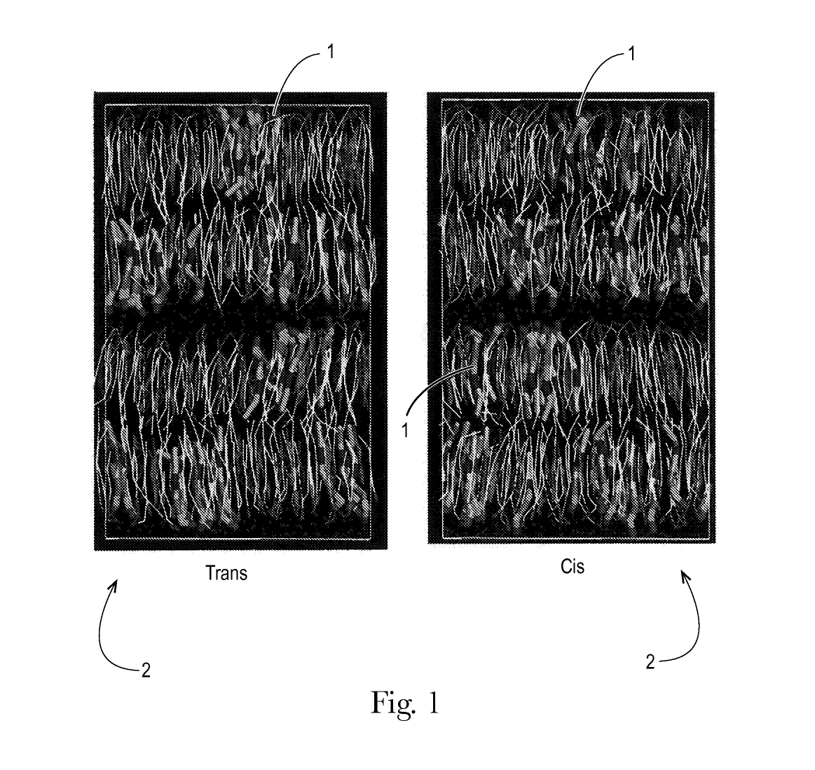

Surprisingly, it has been found there are materials which impact the lipid bilayer allowing for improvement of its barrier function and leading to better skin hydration. Moreover, it has been found these materials can be adequately delivered through a personal care composition. Without being limited by theory, it is believed lipid bilayer structurants, like glyceryl monooleate 1 and glyceryl monostearate, penetrate into the lipid bilayer 2 and act as structurants for the lipid bilayer (see FIG. 1), helping to improve the organization of the layer, and thus enhancing the skin's natural ability to hold on to moisture, improving hydration.

Improvements in skin hydration from use of the compositions described herein can be measured using known techniques, including for example, a Corneometer, which can measure moisture level. Typical Corneometer Units for skin hydration range from about 15-20, wherein the higher the value the higher the level of hydration; and the lower the value, the lower the level of hydration. For example, see Table 1 below. Comparative example A illustrates a benefit from applying a benefit agent containing composition on the skin, which, in and of itself, provides an improvement to skin hydration of 1.05 units from the water control. However, as you can see from Inventive Example B, the replacement of 1% of the benefit agent from Comparative Example A with glyceryl monooleate, a lipid bilayer structurant, resulted in a significant improvement in corneometer reading of 2.08 units from the baseline, which is one full unit higher than the benefit agent alone without glyceryl monooleate (1.05 vs. 2.08). Additionally, inventive Examples C and D contained 0.1% to 0.2% glyceryl monooleate and also showed a significant improvement of 1.31, and 1.76 respectively vs. water control. The corneometer results of inventive Examples (B, C, D) were statistically significant at 95% confidence level.

The corneometer results at 24 hours after one product treatment are also shown below for Comparative Commercial Body wash with soybean oil, Inventive Example E, and Inventive Example F. The commercial product containing soybean oil showed an increase of about 0.54 units from the water control. The inventive Examples E showed an increase of about 1.44 units from the water control. The Inventive Example F showed an increase of about 1.67 units. The corneometer results of inventive Examples E and F were statistically significant at 95% confidence level vs. water control.

TABLE 1

| | Change of Adjusted Corneometer |
|---|---|
| | Mean from Water Control |
| Treatment | At 24 hours after one application |
| Water Control | — |
| Comparative - Commercial Body | +0.54 |
| Wash Containing Soybean Oil | |
| Comparative - Example A | +1.05 |
| Inventive - Example C | +1.31 |
| Inventive Example E | +1.44 |
| Inventive Example F | +1.67 |
| Inventive - Example D | +1.76 |
| Inventive - Example B | +2.08 |

Thus, the application of a personal care composition with a lipid bilayer structurant, like glyceryl monooleate or glyceryl monostearate, will improve hydration of the skin as compared to the water control at 24 hours after one application. Skin hydration can improve, for example, by about 1.2 Corneometer Units or more; by about 1.4 Corneometer Units or more; by about 1.6 Corneometer Units or more; by about 1.8 Corneometer Units or more; or by about 2.0 Corneometer Units or more; where the personal care composition includes a benefit phase with a lipid bilayer structurant. The lipid bilayer structurant may comprise, for example, at least one of glyceryl monooleate and glyceryl monostearate.

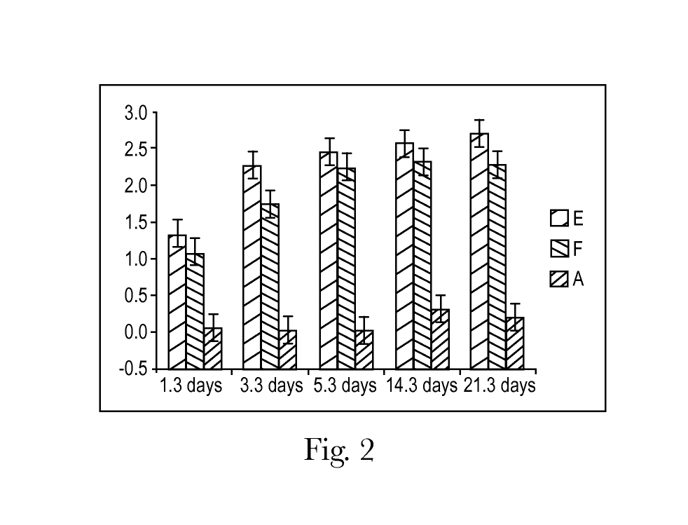

Additionally, as shown in FIG. 2, a composition with a lipid bilayer structurant can also improve the dry skin grade. Column E, which is representative of Inventive Example G which includes 2% glyceryl monooleate and petrolatum in the benefit phase, and is shown in the first column for each day designation, has a higher dry skin grade improvement at all measured points versus Column F (representative of Comparative Example B which has only petrolatum in the benefit phase and is the second column for each day designation) and Column A (which is the water control and is the third column for each day designation).

Thus, the application of a personal cleansing composition with a lipid bilayer structurant, like glyceryl monooleate, glyceryl monostearate, or glyceryl monolaurate, will improve the dry skin grade of the skin as compared to the water control at various time points. Dry skin grade can improve by about 0.1 units or more; by about 0.2 units or more; by about 0.3 units or more; by about 0.5 units or more; or by about 1.0 units or more; where the personal care composition includes a benefit phase with a lipid bilayer structurant. The lipid bilayer structurant may comprise, for example, at least one of glyceryl monooleate and glyceryl monostearate. Dry skin grade can be measured according to the method below.

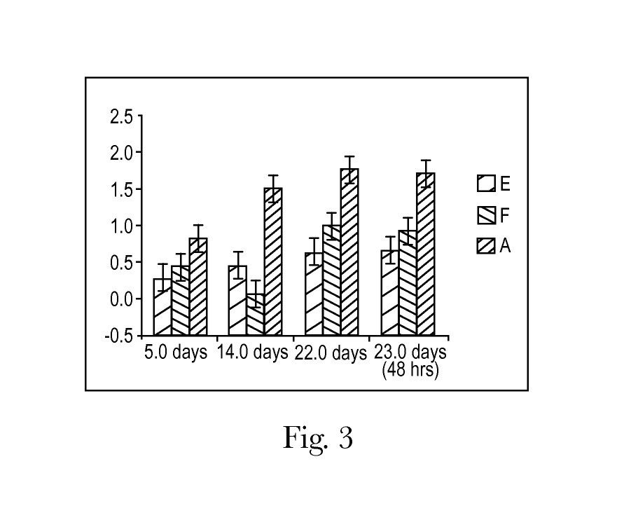

Moreover, as seen in FIG. 3, a composition with a lipid bilayer structurant can also decrease the transepidermal water loss (TEWL). Column E, which represents Inventive Example G and is shown in the first column for each day designation, has a lower TEWL measurement at five days, twenty-two days, and twenty-three days versus Column F, representative of Comparative Composition B, and versus Column A representative of Composition A which is the water control.

Thus, the application of a personal cleansing composition with a lipid bilayer structurant, like glyceryl monooleate, glyceryl monostearate, or glyceryl monolaurate, will improve the TEWL of the skin as compared to the water control at various time points. TEWL can improve by about 0.1 units or more; by about 0.2 units or more; by about 0.3 units or more; by about 0.5 units or more; or by about 1.0 unit or more; where the personal care composition includes a benefit phase with a lipid bilayer structurant. The lipid bilayer structurant may comprise, for example, at least one of glyceryl monooleate and glyceryl monostearate, for example at least one of glyceryl monooleate and glyceryl monostearate. TEWL can be measured according to the method below.

As noted herein, a personal care composition can include a cleansing phase and a benefit phase. The cleansing phase may structured. The cleansing phase and the benefit phase can be in physical contact. A personal care composition can be a multiphase personal care composition where the cleansing phase and the benefit phase can be blended or mixed to a significant degree, but still be physically distinct such that the physical distinctiveness is undetectable to the naked eye. The personal care composition can be a multiphase personal care composition where the cleansing phase and the benefit phase can be made to occupy separate but distinct physical spaces inside a package in which the phases can be stored. For example, the cleansing phase and the benefit phase can be stored such that the phases are not in direct contact with one another. The personal care composition can be a multiphase personal care composition where the cleansing phase and the benefit phase are in physical contact and can have a striped or marbled configuration. For example, one blended multiphase personal care composition can be stacked as stripes with another blended multiphase personal care composition. As an additional example, blended multiphase personal care compositions distinguishable by color can be stacked as stripes wherein the blended multiphase personal care compositions can be otherwise similar.

A. Cleansing Phase

The cleansing phase comprises at least one surfactant. A cleansing phase can include from about 1% to about 20%, from about 2% to about 15%, or from about 5% to about 10%, by weight of the personal care composition, of a surfactant. Suitable surfactants are described in McCutcheon's, Detergents and Emulsifiers, North American edition (1986), published by allured Publishing Corporation; and McCutcheon's, Functional Materials, North American Edition (1992); and in U.S. Pat. No. 3,929,678 issued to Laughlin, et al on Dec. 30, 1975. The personal care composition may comprise, for example, a surfactant selected from the group consisting of: anionic, non-ionic, cationic, zwitterionic, amphoteric, and combinations thereof.

The surfactant may comprise an anionic surfactant. For example, the surfactant may comprise a linear anionic surfactant. These can include, for example, ammonium laureth sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, and combinations thereof. The personal care composition can be optionally free of sodium lauryl sulfate, hereinafter SLS.

The surfactant may comprise a branched anionic surfactant. Some examples of branched anionic surfactants are described in a commonly owned, U.S. Publication No. 2006/0276357. Branched anionic surfactants include, but are not limited to, the following: sodium trideceth sulfate, sodium tridecyl sulfate, sodium C12-13 alkyl sulfate, and C12-13 pareth sulfate, sodium C12-13 pareth-n sulfate, and combinations thereof.

In one example, the anionic surfactant comprises STnS, wherein n can define average moles of ethoxylation. A cleansing phase can include, for example, from about 5% to about 20%, by weight of the personal care composition of STnS. n can range from about 0 to about 3, from about 0.5 to about 2.7, from about 1.1 to about 2.5, from about 1.8 to about 2.2, or n can be about 2. When n can be less than 3, STnS can provide improved stability, improved compatibility of benefit agents within the personal care compositions, and increased mildness of the personal care compositions, such described benefits of STnS are disclosed in U.S. Patent Application Publication No. 2012/0009285.

In one aspect, the personal care compositions of the present invention may comprise an amphoteric surfactant. Suitable amphoteric surfactants include those that are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecyl-aminopropionate, sodium 3-dodecylaminopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and the products described in U.S. Pat. No. 2,528,378. In one aspect, the personal care composition can comprise an amphoteric surfactant that is selected from the group consisting of sodium lauroamphoacetate, sodium cocoamphoactetate, disodium lauroamphoacetate, disodium cocodiamphoacetate, and mixtures thereof. Moreover, amphoacetates and diamphoacetates can also be used.

Zwitterionic surfactants suitable for use include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. Zwitterionic surfactants suitable for use in the multiphase, personal care composition include betaines, including cocoamidopropyl betaine.

Further, the cleansing phase can comprise a structuring system, wherein the structuring system can comprise an associative polymer, a non-associative polymer, and/or an electrolyte. The structuring system can comprise, for example, from about 0.01% to about 5%, from about 0.05% to about 1%, from about 0.07% to about 0.5%, or from about 0.1% to about 0.3%, by weight of the personal care composition, of a non-associative polymer. The structuring system can comprise, for example, from about 0.001% to about 5; from about 0.005% to about 0.5%, from about 0.007% to about 0.05%, from about 0.008% to about 0.04%; or from about 0.01% to about 0.03%, by weight of the personal care composition, of an associative polymer. The stability of a personal care composition can be maintained or enhanced even with the reduction of associative polymer with the addition of a non-associative polymer.

Such associative polymers can be a crosslinked, alkali swellable, associative polymer comprising acidic monomers and associative monomers with hydrophobic end groups, whereby the associative polymer comprises a percentage hydrophobic modification and a hydrophobic side chain comprising alkyl functional groups. Without intending to be limited by theory, it is believed the acidic monomers can contribute to an ability of the associative polymer to swell in water upon neutralization of acidic groups; and associative monomers anchor the associative polymer into structured surfactant hydrophobic domains, e.g., lamellae, to confer structure to the surfactant phase and keep the associative polymer from collapsing and losing effectiveness in a presence of an electrolyte. The crosslinked, associative polymer can comprise a percentage hydrophobic modification, which is a mole percentage of monomers expressed as a percentage of a total number of all monomers in a polymer backbone, including both acidic and other non-acidic monomers. Percentage hydrophobic modification of the associative polymer, hereafter % HM, can be determined by the ratio of monomers added during synthesis, or by analytical techniques such as proton nuclear magnetic resonance (NMR). Associative alkyl side chains can comprise, for example, butyl, propyl, stearyl, steareth, cetyl, lauryl, laureth, octyl, behenyl, beheneth, steareth, or other linear, branched, saturated, or unsaturated alkyl or alketh hydrocarbon side chains.

An associative polymer can include, for example, AQUPEC® SER-300 made by Sumitomo Seika of Japan, which is an acrylate/C₁₀-C₃₀ alkyl acrylate cross-polymer and comprises stearyl side chains with less than about 1% HM. Associative polymers can comprise about C₁₆ (cetyl) alkyl hydrophobic side chains with about 0.7% hydrophobic modification, but a percentage hydrophobic modification can be up to an aqueous solubility limit in surfactant compositions (e.g., up to 2%, 5%, or 10%). Other associative polymers can include stearyl, octyl, decyl and lauryl side chains, alkyl acrylate polymers, polyacrylates, hydrophobically-modified polysaccharides, hydrophobically-modified urethanes, AQUPEC® SER-150 (acrylate/C₁₀-C₃₀ alkyl acrylate cross-polymer) comprising about C₁₈ (stearyl) side chains and about 0.4% HM, and AQUPEC® HV-701EDR which comprises about C₈ (octyl) side chains and about 3.5% HM, and mixtures thereof. The associative polymer can be, for example, Stabylen 30 manufactured by 3V Sigma S.p.A., which has branched isodecanoate hydrophobic associative side chains.

As set forth above, the cleansing phase of a personal care composition can further include a non-associative polymer. Suitable non-associative polymers can include water-dispersible polymers with relatively uniform hydrophilic backbone lacking hydrophobic groups. Examples of non-associative polymers can include biopolymer polysaccharides (e.g., xanthan gum, gellan gum), cellulosic polysaccharides (e.g., carboxymethyl cellulose, carboxymethyl hydroxyethyl cellulose), other polysaccharides (e.g., guar gum, hydroxypropyl guar, and sodium alginate), and synthetic hydrocarbon polymers (e.g., polyacrylamide and copolymers, polyethylene oxide, polyacrylic acid copolymers).

Suitable electrolytes can includes an anion such as phosphate, chloride, sulfate, citrate, and mixtures thereof and a cation such as sodium, ammonium, potassium, magnesium, and mixtures thereof. For example, suitable electrolytes can include sodium chloride, ammonium chloride, sodium sulfate, ammonium sulfate, and mixtures thereof. Other suitable electrolytes are described in U.S. Patent Application Publication No. 2012/0009285.

Personal care compositions can additionally comprise an organic cationic deposition polymer in one or more phases as a deposition aid for benefit agents described herein. Suitable cationic deposition polymers can contain cationic nitrogen-containing moieties such as quaternary moieties. Non-limiting examples of cationic deposition polymers can include polysaccharide polymers, such as cationic cellulose derivatives. Cationic cellulose polymers can be salts of hydroxyethyl cellulose reacted with trimethyl ammonium substituted epoxide, referred to in the industry (CTFA) as Polyquaternium 10, which can be available from Amerchol Corp. (Edison, N.J.) in their Polymer KG, JR, and LR series of polymers. Other suitable cationic deposition polymers can include cationic guar gum derivatives, such as guar hydroxypropyltrimonium chloride, specific examples of which can include the Jaguar series commercially available from Rhodia Inc. and N-Hance polymer series commercially available from Aqualon. Deposition polymers can have, for example, a cationic charge density from about 0.8 meq/g to about 2.0 meq/g or from about 1.0 meq/g to about 1.5 meq/g.

The cleansing phase of the personal care composition can also comprise water. The structured cleansing phase of the personal care composition can comprise, for example, from about 10% to about 90%, from about 40% to about 85%, or from about 60% to about 80%, by weight of water.

B. Benefit Phase

As noted herein, personal care compositions can include a benefit phase. The benefit phase can be hydrophobic and/or anhydrous. The benefit phase comprises a lipid bilayer structurant.

The benefit phase can comprise from about 0.05% to about 10%, by weight of the personal care composition, of a lipid bilayer structurant. Moreover, the benefit phase can comprise, for example, from about 0.05% to about 5%; from about 0.05% to about 2.5%, from about 0.05% to about 2.0%, from about 0.05% to about 1.0%, or any combination thereof, by weight of the personal care composition, of a lipid bilayer structurant. Suitable lipid bilayer structurants include, for example, glyceryl monooleate, glyceryl monostearate, glyceryl monolaurate, glyceryl dilaurate, and combinations thereof. In one example, the lipid bilayer structurant comprises glyceryl monooleate, glyceryl monostearate, glyceryl monolaurate, or a combination thereof. In another example, the lipid bilayer structurant comprises glyceryl monooleate.

The benefit phase can also include one or more benefit agents. In particular, the benefit phase can comprise from about 0.1% to about 50%, by weight of the personal care composition, of a benefit agent. The benefit phase can also include from about 0.5% to about 20%, by weight of the personal care composition, of the benefit agent. Examples of the benefit agent can include petrolatum, sucrose polyester, mineral oil, natural oils (e.g., soybean oil), and mixtures thereof. Other suitable benefit agents are described in U.S. Patent Application Publication No. 2012/0009285.

Benefit agents can include water insoluble or hydrophobic benefit agents. Additional examples of benefit agents can include SEFOSE®, lanolin, lanolin derivatives, lanolin esters, lanolin oil, natural waxes, synthetic waxes, volatile organosiloxanes, derivatives of volatile organosiloxanes, non-volatile organosiloxanes, derivatives of non-volatile organosiloxanes, natural triglycerides, synthetic triglycerides, and combinations thereof.

SEFOSE® includes one or more types of sucrose polyesters. Sucrose polyesters are derived from a natural resource and therefore, the use of sucrose polyesters as the benefit agents can result in a positive environmental impact. Sucrose polyesters are polyester materials having multiple substitution positions around the sucrose backbone coupled with the chain length, saturation, and derivation variables of the fatty chains. Such sucrose polyesters can have an esterification (“IBAR”) of greater than about 5. The sucrose polyester may have, for example, an IBAR of from about 5 to about 8; from about 5 to about 7, an IBAR of about 6 or an IBAR of about 8. As sucrose polyesters are derived from a natural resource, a distribution in the IBAR and chain length may exist. For example a sucrose polyester having an IBAR of 6, may contain a mixture of mostly IBAR of about 6, with some IBAR of about 5 and some IBAR of about 7. Additionally, such sucrose polyesters may have a saturation or iodine value (“IV”) of about 3 to about 140, about 10 to about 120, or about 20 to 100. Further, such sucrose polyesters can have a chain length of about C₁₂ to C₂₀.

Non-limiting examples of sucrose polyesters suitable for use include SEFOSE® 1618S, SEFOSE® 1618U, SEFOSE® 1618H, Sefa Soyate IMF 40, Sefa Soyate LP426, SEFOSE® 2275, SEFOSE® C1695, SEFOSE® C18:0 95, SEFOSE® C1495, SEFOSE® 1618H B6, SEFOSE® 1618S B6, SEFOSE® 1618U B6, Sefa Cottonate, SEFOSE® C1295, Sefa C895, Sefa C1095, SEFOSE® 1618S B4.5, all available from The Procter and Gamble Co. of Cincinnati, Ohio.

Non-limiting examples of glycerides suitable for use as hydrophobic benefit agents herein can include castor oil, safflower oil, corn oil, walnut oil, peanut oil, olive oil, cod liver oil, almond oil, avocado oil, palm oil, sesame oil, soybean oil, vegetable oils, sunflower seed oil, vegetable oil derivatives, coconut oil and derivatized coconut oil, cottonseed oil and derivatized cottonseed oil, jojoba oil, cocoa butter, petrolatum, mineral oil, and combinations thereof.

Non-limiting examples of alkyl esters suitable for use as hydrophobic benefit agents herein can include isopropyl esters of fatty acids and long chain esters of long chain (i.e. C₁₀-C₂₄) fatty acids, e.g., cetyl ricinoleate, non-limiting examples of which can include isopropyl palmitate, isopropyl myristate, cetyl riconoleate, and stearyl riconoleate. Other examples can include hexyl laurate, isohexyl laurate, myristyl myristate, isohexyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, acyl isononanoate lauryl lactate, myristyl lactate, cetyl lactate, and combinations thereof.

Non-limiting examples of alkenyl esters suitable for use as hydrophobic benefit agents herein can include oleyl myristate, oleyl stearate, oleyl oleate, and combinations thereof.

Non-limiting examples of polyglycerin fatty acid esters suitable for use as hydrophobic benefit agents herein can include decaglyceryl distearate, decaglyceryl diisostearate, decaglyceryl monomyriate, decaglyceryl monolaurate, hexaglyceryl monooleate, and combinations thereof.

Non-limiting examples of silicone oils suitable for use as hydrophobic benefit agents herein can include dimethicone copolyol, dimethylpolysiloxane, diethylpolysiloxane, mixed C₁-C₃₀ alkyl polysiloxanes, phenyl dimethicone, dimethiconol, and combinations thereof. Non-limiting examples of silicone oils useful herein are described in U.S. Pat. No. 5,011,681. Still other suitable hydrophobic skin benefit agents can include milk triglycerides (e.g., hydroxylated milk glyceride) and polyol fatty acid polyesters.

Additional optional ingredients can also be added to the personal care composition for treatment of the skin, or to modify the aesthetics of the personal care composition as is the case with perfumes, colorants, dyes or the like. Optional materials useful in products herein can be categorized or described by their cosmetic and/or therapeutic benefit or their postulated mode of action or function. However, it can be understood that actives and other materials useful herein can, in some instances, provide more than one cosmetic and/or therapeutic benefit or function or operate via more than one mode of action. Therefore, classifications herein can be made for convenience and cannot be intended to limit an ingredient to particularly stated application or applications listed. A precise nature of these optional materials, and levels of incorporation thereof, will depend on the physical form of the composition and the nature of the cleansing operation for which it is to be used. Optional materials can usually be formulated at about 6% or less, about 5% or less, about 4% or less, about 3% or less, about 2% or less, about 1% or less, about 0.5% or less, about 0.25% or less, about 0.1% or less, about 0.01% or less, or about 0.005% or less of the personal care composition.

To further improve stability under stressful conditions such as high temperature and vibration, densities of separate phases can be adjusted such that they can be substantially equal. To achieve this, low density microspheres can be added to one or more phases of the personal care composition. Examples of personal care compositions that comprise low density microspheres are described in a patent application published on May 13, 2004 under U.S. Patent Publication No. 2004/0092415A1 entitled “Striped Liquid Personal Cleansing Compositions Containing A Cleansing Phase and A Separate Phase with Improved Stability,” filed on Oct. 31, 2003 by Focht, et al.

### III. Methods Relating to the Use of Personal Care Compositions

Personal care compositions as described herein can provide a multitude of benefits. For example, a method of enhancing skin hydration includes formulating or providing a personal care composition comprising a cleansing phase and a benefit phase. The benefit phase comprises a benefit agent and a lipid bilayer structurant. An individual is instructed to apply the composition to their skin. Skin hydration is improved by about 1.2 corneometer units, as compared to the water control, at 24 hours after one application. Said another way, the hydration effect compared to the water control is such that said hydration effect is 1.2 Corneometer Units above the water control at 24 hours after one application. The lipid bilayer structurant may comprise glyceryl monooleate, glyceryl monostearate, or a combination thereof. The method may further comprise instructing the individual to rinse the personal care composition from the skin.

Also included is a method of enhancing skin hydration including applying a personal care composition to the skin of an individual. The benefit phase comprises a benefit agent and a lipid bilayer structurant. Skin hydration is improved by about 1.2 corneometer units, as compared to the water control, at 24 hours after one application. Said another way, the hydration effect compared to the water control is such that said hydration effect is 1.2 Corneometer Units above the water control at 24 hours after one application. The lipid bilayer structurant may comprise glyceryl monooleate, glyceryl monostearate, or a combination thereof. The method may further comprise instructing the individual to rinse the personal care composition from the skin.

Also included is a method of enhancing skin hydration comprising formulating or providing a personal care composition and applying the personal care composition to skin of an individual. The personal care composition may comprise a cleansing phase and a benefit phase. The benefit phase may comprise a benefit agent and a lipid bilayer structurant. Skin hydration is improved by about 1.2 corneometer units, as compared to the water control, at 24 hours after one application. Said another way, the hydration effect compared to the water control is such that said hydration effect is 1.2 Corneometer Units above the water control at 24 hours after one application. The lipid bilayer structurant may comprise glyceryl monooleate, glyceryl monostearate, or a combination thereof. The method may further comprise rinsing the personal care composition from the skin.

Also included is a method of improving dry skin which includes applying a personal care composition to the skin on an individual, wherein the composition comprises a cleansing phase and a benefit phase. The benefit phase comprises a benefit agent and a lipid bilayer structurant. Dry skin grade improves by about 0.1 units, as compared to the water control. Said another way, the dry skin effect compared to the water control is such that said dry skin effect is 0.1 units above the water control. The lipid bilayer structurant may comprise glyceryl monooleate, glyceryl monostearate, or a combination thereof.

Additionally included is a method of improving dry skin which includes formulating or providing a personal care composition comprising a cleansing phase and a benefit phase. The benefit phase comprises a benefit agent and a lipid bilayer structurant. An individual is instructed to apply the composition to their skin. Dry skin grade is improved by about 0.1 units, as compared to the water control. Said another way, the dry skin effect compared to the water control is such that said dry skin effect is 0.1 units above the water control. The lipid bilayer structurant may comprise glyceryl monooleate, glyceryl monostearate, or a combination thereof. The method may further comprise instructing the individual to rinse the personal care composition from the skin.

Also included is a method of improving dry skin comprising formulating or providing a personal care composition and applying the personal care composition to skin of an individual. The personal care composition may comprise a cleansing phase and a benefit phase. The benefit phase may comprise a benefit agent and a lipid bilayer structurant. Dry skin grade is improved by about 0.1 units, as compared to the water control. Said another way, the dry skin effect compared to the water control is such that said dry skin effect is 0.1 units above the water control. The lipid bilayer structurant may comprise glyceryl monooleate, glyceryl monostearate, or a combination thereof. The method may further comprise rinsing the personal care composition from the skin.

Also included is a method of decreasing transepidermal water loss which includes applying a personal care composition to the skin on an individual, wherein the composition comprises a cleansing phase and a benefit phase. The benefit phase comprises a benefit agent and a lipid bilayer structurant. TEWL improves by about 0.1 units, as compared to the water control. Said another way, the transepidermal water loss compared to the water control is such that said TEWL improves by at least 0.1 units, as compared to the water control. The lipid bilayer structurant may comprise glyceryl monooleate, glyceryl monostearate, or a combination thereof.

Additionally included is a method of decreasing transepidermal water loss which includes formulating or providing a personal care composition comprising a cleansing phase and a benefit phase. The benefit phase comprises a benefit agent and a lipid bilayer structurant. An individual is instructed to apply the composition to their skin. TEWL is improved by about 0.1 units, as compared to the water control. Said another way, the transepidermal water loss compared to the water control is such that said TEWL improves by at least 0.1 units, as compared to the water control. The lipid bilayer structurant may comprise glyceryl monooleate, glyceryl monostearate, or a combination thereof. The method may further comprise instructing the individual to rinse the personal care composition from the skin.

Also included is a method of decreasing transepidermal water loss comprising formulating or providing a personal care composition and applying the personal care composition to skin of an individual. The personal care composition may comprise a cleansing phase and a benefit phase. The benefit phase may comprise a benefit agent and a lipid bilayer structurant. TEWL is improved by about 0.1 units, as compared to the water control. Said another way, the transepidermal water loss compared to the water control is such that said TEWL improves by at least 0.1 units, as compared to the water control. The lipid bilayer structurant may comprise glyceryl monooleate, glyceryl monostearate, or a combination thereof. The method may further comprise rinsing the personal care composition from the skin.

For the sake of brevity, only a few particular composition components are set out in this section. However, it is envisioned the personal care composition can have any of the components of the personal care compositions as described more fully above.

### IV. Test Methods

A. T-Bar Viscosity Method

The viscosity of a personal care composition can be assessed by the T-Bar Viscosity Method. The apparatus for T-Bar measurements includes a Brookfield DV-II+ Pro Viscometer with Helipath Accessory; a chuck, weight and closer assembly for T-bar attachment; a T-bar Spindle D, a personal computer with Rheocalc software from Brookfield, and a cable connecting a Brookfield Viscometer to a computer. First, weigh 80 grams of a personal care composition in a 4-oz. glass jar. Measure a T-bar viscosity by carefully dropping the T-Bar Spindle to an interior bottom of the glass jar and set the Helipath stand to travel in an upward direction. Open the Rheocalc software and set the following data acquisition parameters: Speed to 5 rpm, Time Wait for Torque to 00:01 (1 second), and Loop Start Count to 100. Start data acquisition and turn on the Helipath stand to travel upward at a speed of 22 mm/minute. The T-Bar viscosity is an average T-Bar viscosity reading between the 10^{th} reading and the 90^{th} reading (the first ten readings and the last ten readings are not used for the average T-Bar viscosity calculation). The T-Bar viscosity reading is provided in cP. After obtaining the initial viscosity reading, place the personal care composition at 50° C. for 10 days for rapid aging. After finishing the stability testing at 50° C., the sample is equilibrated at 25° C. for 24 hours. Then repeat viscosity measurement to obtain final viscosity. Measure percent change of the initial viscosity from the final viscosity measurement to obtain the percent change in viscosity.

B. Zero Shear Viscosity

The Zero Shear Viscosity of a material which is a phase or a composition of the personal care composition, can be measured either prior to combining in the personal care composition, after preparing a composition, or first separating a phase or component from a composition by suitable physical separation means, such as centrifugation, pipetting, cutting away mechanically, rinsing, filtering, or other separation means.

A controlled stress rheometer such as a TA Instruments AR2000Rheometer is used to determine the Zero Shear Viscosity. The determination is performed at 25° C. with a 4 cm diameter parallel plate measuring system and a 1 mm gap. The geometry has a shear stress factor of 79580 m-3 to convert torque obtained to stress. Serrated plates can be used to obtain consistent results when slip occurs.

First, material is positioned on a rheometer base plate; the measurement geometry (upper plate) is moved into position 1.1 mm above the base plate. Excess material at the geometry edge is removed by scraping after locking the geometry. The geometry is then moved to the target 1 mm position above the base plate and a pause of about 2 minutes is allowed to allow loading stresses to relax. This loading procedure ensures no tangential stresses are loaded at the measurement onset, which can influence results obtained. If the material comprises particles discernible to the eye or by feel (beads, e.g.) which are larger than about 150 microns in number average diameter, the gap setting between the base plate and upper plate is increased to the smaller of 4 mm or 8-fold the diameter of the 95^{th} volume percentile particle diameter. If a phase has any particle larger than 5 mm in any dimension, the particles are removed prior to the measurement.

The measurement is performed by applying a continuous shear stress ramp from 0.1 Pa to 1,000 Pa over a time interval of 4 minutes using a logarithmic progression, i.e., measurement points evenly spaced on a logarithmic scale. Thirty measurement points per decade of stress increase are obtained. If the measurement result is incomplete, for example, if material is observed to flow from the gap, results obtained are evaluated with incomplete data points excluded. If there are insufficient points to obtain an accurate measurement, the measurement is repeated with increased number of sample points.

The Zero Shear Viscosity is obtained by taking a first median value of viscosity in Pascal-seconds (Pa-s) for viscosity data obtained between and including 0.1 Pa and a point where viscosity begins to steeply decline. After taking the first median viscosity, all viscosity values greater than 5-fold the first median value and less than 0.2× the median value are excluded, and a second median viscosity value is obtained of the same viscosity data, excluding the indicated data points. The second median viscosity so obtained is the Zero Shear Viscosity.

As set forth above, a structured cleansing phase can be considered to be structured if the structured cleansing phase has a Zero Shear Viscosity of about 200 Pa-s or more, about 500 Pa-s or more, about 1,000 Pa-s or more, about 1,500 Pa-s or more, or about 2,000 Pa-s or more.

C. Dry Skin Grade Screen and Application of Materials for Corneometer and TEWL Testing

Test subjects are screened for dry skin grade of 2.5-4.0 by trained expert graders following guidelines below. Prior to the study, subjects participate in a washout period for seven days, in which the subjects only use soap that is provided to them (e.g., soap including shea butter and no beads) and abstain from washing their legs with any other products. Subjects are also instructed to abstain from applying any leave-on products to their legs during the pre-study washout period.

Visual evaluations will be done with the aid of an Illuminated Magnifying Lamp which provides 2.75× magnification and which has a shadow-free circular fluorescent light source (General Electric Cool White, 22 watt 8″ Circline). At least 36 subjects are needed to obtain sufficient replicates for each treatment. Table 2 shows a grading scale for dry skin and lists the redness and dryness characteristics associated with each grade.

TABLE 2

| Grade* | Redness | Dryness** |
|---|---|---|
| 0.0 | No redness | Perfect skin |
| 1.0 | Barely | Patches of checking and/or slight powderiness, |
| | detectable | occasional patches of small scales maybe seen, |
| | redness | distribution generalized |
| 2.0 | Slight | Generalized slight powderiness, early cracking, or |
| | redness | occasional small lifting scales may be present |
| 3.0 | Moderate | Generalized moderate powderiness and/or heavy |
| | redness | cracking and lifting scales |
| 4.0 | Heavy or | Generalized heavy powderiness and/or heavy |
| | substantial | cracking and lifting scales |
| | redness | |
| 5.0 | Severe | Generalized high cracking and lifting scales, |
| | redness | eczematous change may be present, but not |
| | | prominent, may see bleeding cracks |
| 6.0 | Extreme | Generalized severe cracking, bleeding cracks and |
| | redness | eczematous changes may be present, large scales |
| | | may be sloughing off |

*Half-unit grades may be used if necessary
**“Generalized” refers to situations where more than 50% of an application area is affected

Before initial visual grading, a clinical assistant will mark 2-7 cm (across)×10 cm (down) treatment sites on an outer portion of the lower legs using a template and a laboratory marking pen (4 corner brackets are sufficient to delineate each area). For assignment of the products, two sites located on the left leg will be numbered L1 and L2, where L1 is the top part of the lower leg nearest the knee, and L2 is the bottom part of the lower leg nearest the ankle. Two sites located on the right leg will be numbered R1 and R2, where R1 is the top part of the lower leg nearest the knee, and R2 is the bottom part of the lower leg nearest the ankle.

To simplify the treatment process, master trays will be prepared for each treatment plan specified in the study randomization. Each master tray will be divided in half, with each half labeled ‘left’ or ‘right’ to indicate which leg it corresponds to, then subdivided into sections for the test products in the order of leg application site. One or more make-up trays can also be prepared for use as needed using individual coded containers, or other appropriate product code indicators, that can be re-arranged according to a given treatment plan.

Trained clinical assistants will wash each subject's lower legs in a controlled manner with assigned treatments once daily for 21 consecutive days. Assignment of test treatments to skin sites on the left and right legs will be designated by study randomization. A target dose of body wash for each site is 10 μL/cm². All body wash products will be dispensed at 0.7 mL dosages. All body wash test products will be drawn up into syringes at the 0.7 mL dosage. A one day supply of syringes for all products may be filled the day before or the day of use. Product that has been transferred to another container and the container itself will be used for one day only (i.e., the day the transfer occurred). All syringe filling operations will be appropriately documented (e.g., product code filled, when filled, initials of person responsible for filling).

The treatment area on the top part of the left leg of the subject is wetted for 5 seconds with 95-100° F. running tap water. The water flow rate is about 1200 mL per minute. For the “No Treatment” site, apply water only. For a treatment site, dispense 0.7 mL of body wash product from the syringe onto the center of the treatment area and place a wet puff over the dispensed product and gently rub the puff back and forth within the treatment site for 10 seconds. Then, allow lather (or water only) to remain on the site for 90 seconds. When residence time for a site has expired, the site is rinsed for 15 seconds under a running tap, taking care not to rinse adjacent sites. After the application area has been rinsed, the area is gently patted dry. Repeat the procedure for the lower part of the left leg, and after completion, use the same procedure for each of the top part of the right leg and the lower part of the right leg.

D. Corneometer Testing

Once the materials are applied as noted above in Section A, improvements in skin hydration can be measured with a Corneometer, while baseline measurements are taken prior to application of materials. In particular, skin hydration based upon measurements of capacitance can be assessed using the Corneometer® 825. Such use of a Corneometer is further described in U.S. patent application Ser. No. 13/007,630. Such measurements can be non-invasive and can be taken in duplicate on each site of the subjects' legs at the following times: At baseline, prior to 1^{st} treatment; 3 hours post 1st 3^{rd}, 5^{th}, 14^{th} and 21^{st} treatments; 24 hours post 4^{th}, 13^{th} and 21^{st}, treatments, 48 hours post 21^{st} treatment after a visual assessment has been completed. Subjects can be acclimated for a minimum of thirty minutes in an environmentally controlled room (maintained at 70° F.±2 and 30-45% relative humidity) prior to the non-invasive instrumental measurements taken on their legs. Data can be recorded electronically using a Sponsor's direct data entry and data capture programs. Measurements can be performed according to a test facility's standard operating procedures and/or the Sponsors Instrument Operation Manual.

The Corneometer values are arbitrary units for electrical impedance. At baseline, for subjects having a dry skin grade from about 2.5 to about 4.0, an adjusted mean of such Corneometer values can typically fall within a range of about 15 to about 20. Higher Corneometer values can correspond to a higher hydration level, and thus, lower Corneometer values can correspond to lower hydration levels.

The instrument should only be operated by trained operators. Further, the same instrument(s) and operator(s) can be used throughout the study. Kimwipes can be used to wipe an end of a probe.

The probe can be wiped with a Kimwipe between each measurement. At the end of an evaluation session, data collected for that period can be backed up according to instructions in the Sponsors Instrument Operation Manual, and a hard copy of the data can be printed.

E. Transepidermal Water Loss (TEWL) Method

Once the materials are applied as noted above in Section A, the step of assessing erythema and/or dryness by objective instrumental measurements may include evaluating the portion of skin with a transepidermal water loss instrument, commercially available from Cortex Technology, Denmark under the trade name TEWL, DermaLab® Evaporimeter. Participants may be conditioned in a temperature and humidity controlled room (73° F.±4° F. (about 23° C.±2.2° C.) and a relative humidity of 50%±10%) for approximately 20 minutes.

### V. Examples

The following examples describe and demonstrate examples within the scope of the invention. The examples are given solely for the purpose of illustration and are not to be construed as limitations of the present invention, as many variations thereof are possible without departing from the spirit and scope of the invention.

| | Comp. | Inv. | Inv. | Inv. | Inv. | Inv. |
|---|---|---|---|---|---|---|
| | Exp. A | Exp. B | Exp. C | Exp. D | Exp. E | Exp. F |
| Surfactant phase | | | | | | |
| Water and minors (ex. | Q.S. | Q.S. | Q.S. | Q.S. | Q.S. | Q.S. |
| fragrance) | | | | | | |
| Sodium Trideceth-2 Sulfate | 7.2 | 7.2 | 7.3 | 7.3 | 7.7 | 7.7 |
| Sodium Lauroamphoacetate | 3.0 | 3.0 | — | — | — | — |
| Cocoamidopropyl Betain | — | — | 2.05 | 2.05 | 2.17 | 2.17 |
| Sodium Chloride | 4.75 | 4.75 | 4.75 | 4.75 | 4.75 | 4.75 |
| Trideceth-3 | 0.9 | 0.9 | 1.4 | 1.4 | 1.32 | 1.32 |
| Guar | 0.53 | 0.53 | 0.37 | 0.37 | 0.39 | 0.39 |
| hydroxypropyltrimonium | | | | | | |
| chloride | | | | | | |
| (N-Hance CG-17 from | | | | | | |
| Aqualon) | | | | | | |
| Sodium Benzoate | 0.28 | 0.28 | 0.28 | 0.28 | 0.28 | 0.28 |
| Methylchloroisothiazoli | 0.037 | 0.037 | 0.037 | 0.037 | 0.037 | 0.037 |
| none/isothiaxolinone | | | | | | |
| (Kathon CG from | | | | | | |
| Rohm & Haas) | | | | | | |
| C10-C30 Alkylacrylates | 0.17 | 0.17 | 0.18 | 0.18 | 0.14 | 0.14 |
| Cross Polymer | | | | | | |
| (Aqupec Ser W-300C | | | | | | |
| from Sumitomo) | | | | | | |
| EDTA | 0.15 | 0.15 | 0.15 | 0.15 | 0.15 | 0.15 |
| Adjust pH to 5.7 with | | | | | | |
| either citric acid or NaOH | | | | | | |
| Benefit Phase | | | | | | |
| Soybean oil | 10.0 | 9.0 | 9.9 | — | 4.95 | — |
| Petrolatum | — | — | — | 9.8 | — | 4.90 |
| Glyceryl monooleate | — | 1.0 | 0.1 | 0.2 | 0.05 | 0.1 |

| Cleansing Phase | Comparative Example B | Inventive Example G |
|---|---|---|
| Water | Q.S. | Q.S. |
| Sodium Lauryl Sulfate | 4.14 | 4.14 |
| Sodium Lauroamphoacetate | 2.47 | 2.47 |
| Sodium Trideceth Sulfate | 4.14 | 4.14 |
| Sodium Chloride | 2.375 | 2.375 |
| Trideceth-3 (Iconol TDA-3) | 1 | 1 |
| Methylchloroisothiazolinone | 0.0007 | 0.0007 |
| (Kathon CG) | | |
| Citric Acid | 0.45 | 0.45 |
| Guar Hydroxypropyltrimonium | 0.3 | 0.3 |
| Chloride (N-Hance 3196) | | |
| Xanthan Gum (Keltrol 1000) | 0.11 | 0.11 |
| Sodium Benzoate | 0.1 | 0.1 |
| PEG-90M (Polyox WSR 301) | 0.075 | 0.075 |
| Disodium EDTA (Dissolvine NA | 0.075 | 0.075 |
| 2X) | | |
| Sodium Hydroxide | 0.0185 | 0.0185 |
| Fragrance | 0.91 | 0.91 |
| Benefit Phase | 0 | 0 |
| Petrolatum | 50 | 49 |
| Glyceryl monooleate | — | 1 |

Comparative Example A can be prepared through a conventional mixing technique. First, prepare a polymer premix by adding Aqupec SER-300C into Trideceth-3 in a container and separately prepare a citric acid premix in another container (made by adding citric acid power into water at 50:50 w/w ratio). Once the two pre-mixes are completed, add water into the main mixing vessel. Then add sodium chloride, guar hydroxypropyltrimonium chloride, sodium lauroamphoaceate, sodium trideceth-2 sulfate, trideceth-3/Aqupec premix (above), sodium benzoate, and EDTA with continuous mixing. Adjust pH to about 5.7 by adding citric acid solution (above) or NaOH solution. Then, add perfume and Kathon. This completes the cleansing phase. Then, add the benefit phase, soybean oil, into the surfactant phase. Keep mixing until homogeneous.

Inventive Example B can be prepared through a conventional mixing technique. First, prepare a polymer premix by adding Aqupec SER-300C into Trideceth-3 in a container and separately prepare a citric acid premix in another container (made by adding citric acid power into water at 50:50 w/w ratio). Once the two pre-mixes are completed, add water into the main mixing vessel. Then add sodium chloride, guar hydroxypropyltrimonium chloride, sodium lauroamphoaceate, sodium trideceth-2 sulfate, trideceth-3/Aqupec premix (above), sodium benzoate, and EDTA with continuous mixing. Adjust pH to about 5.7 by adding citric acid solution (above) or NaOH solution. Then, add perfume and Kathon. This completes the cleansing phase. In a separate lipid container, add soybean oil and heat to about 50 C, then add glyceryl monooleate into soybean oil with mixing. Then, add the hot soybean oil/glyceryl monooleate lipid phase into the cleansing phase. Keep mixing until homogeneous.

Inventive Example C and E can be prepared through a conventional mixing technique. First, prepare a polymer premix by adding Aqupec SER-300C into Trideceth-3 in a container and separately prepare a citric acid premix in another container (made by adding citric acid power into water at 50:50 w/w ratio). Once the two pre-mixes are completed, add water into the main mixing vessel. Then add sodium chloride, guar hydroxypropyltrimonium chloride, cocamidopropyl betain, sodium trideceth-2 sulfate, trideceth-3/Aqupec premix, sodium benzoate, EDTA with continuous mixing. Adjust pH to about 5.7 by adding citric acid solution or NaOH solution. Then, add perfume and Kathon. In a separate lipid container, add soybean oil and heat to about 50 C, then add glyceryl monooleate into soybean oil with mixing. Then, add soybean oil/glyceryl monooleate lipid phase into the cleansing phase. Keep mixing until homogeneous.

Inventive Example D and F can be prepared through a conventional mixing technique. First, prepare a polymer premix by adding Aqupec SER-300C into Trideceth-3 in a container and separately prepare a citric acid premix in another container (made by adding citric acid power into water at 50:50 w/w ratio). Once the two pre-mixes are completed, add water into the main mixing vessel. Then add sodium chloride, guar hydroxypropyltrimonium chloride, cocamidopropyl betain, sodium trideceth-2 sulfate, trideceth-3/Aqupec premix, sodium benzoate, EDTA with continuous mixing. Adjust pH to about 5.7 by adding citric acid solution or NaOH solution. Then, add perfume and Kathon. In a separate lipid container, add petrolatum and heat to about 80 C, then add glyceryl monooleate into petrolatum with mixing. Cool the lipid phase to about 60 C with mixing. Then, add petrolatum/glyceryl monooleate lipid phase into the cleansing phase. Keep mixing until homogeneous.

Comparative Example B can be prepared through a conventional mixing technique. Add water into the main mixing vessel. Add sodium chloride, N-Hance 3196, Sodium Lauroamphoacetate, Sodium Lauryl Sulfate, Sodium Trideceth-3 Sulfate, Trideceth-3, Keltrol 1000, PEG-90M, sodium benzoate, EDTA. Adjust the pH to about 5.7 by adding citric acid solution. Then, add perfume and Kathon. This completes the cleansing phase. Then, heat the benefit phase, petrolatum, to about 60 C while mixing. Then, add the petrolatum into the surfactant phase. Keep mixing until homogeneous.

Inventive Example G can be prepared through a conventional mixing technique. Add water into the main mixing vessel. Add sodium chloride, N-Hance 3196, Sodium Lauroamphoacetate, Sodium Lauryl Sulfate, Sodium Trideceth-3 Sulfate, Trideceth-3, Keltrol 1000, PEG-90M, sodium benzoate, EDTA. Adjust the pH to about 5.7 by adding citric acid solution. Then, add perfume and Kathon. This completes the cleansing phase. Then, heat the benefit phase, petrolatum, to about 60 C, and add glyceryl monooleate while mixing. Then, add the petrolatum/glyceryl monooleate into the surfactant phase. Keep mixing until homogeneous.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular examples of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of enhancing skin hydration, the method comprising:
applying a personal cleansing composition to skin, wherein the personal cleansing composition, comprises:
i. a cleansing phase; and
ii. a benefit phase comprising a hydrophobic benefit agent and a lipid bilayer structurant; and
rinsing the personal cleansing composition from the skin,
wherein skin hydration improves by about 1.2 Corneometer Units or more, as compared to a water control, at 24 hours after one application.

2. The method of claim 1, wherein the benefit phase comprises from about 0.05% to about 10%, by weight of the personal cleansing composition, of the lipid bilayer structurant which comprises glyceryl monooleate, glyceryl monostearate, glyceryl monolaurate, or a combination thereof.

3. The method of claim 2, wherein the benefit phase comprises from about 0.05% to about 5% of the lipid bilayer structurant, by weight of the personal cleansing composition.

4. The method of claim 2, wherein the benefit phase comprises from about 0.05% to about 1% of the lipid bilayer structurant, by weight of the personal cleansing composition.

5. The method of claim 1, wherein the benefit agent comprises petrolatum, soybean oil, sucrose polyester, mineral oil, or a combination thereof.

6. The method of claim 1, wherein the skin hydration improves by about 1.4 Corneometer units or more.

7. The method of claim 1, wherein the benefit phase is free of surfactant.

8. The method of claim 1, wherein the benefit phase is anhydrous.

9. A method of enhancing skin hydration, the method comprising:
formulating a personal cleansing composition, wherein the personal cleansing composition, comprises:
i. a cleansing phase; and
ii. a benefit phase comprising a hydrophobic benefit agent and a lipid bilayer structurant; and
instructing an individual to apply the personal cleansing composition to skin,
wherein skin hydration improves by about 1.2 Corneometer Units or more, as compared to a water control, at 24 hours after one application.

10. The method of claim 9, wherein the individual is further instructed to rinse the personal cleansing composition from the skin.

11. The method of claim 9, wherein the cleansing phase and benefit phase are blended.

12. The method of claim 9, wherein the cleansing phase and benefit phase are patterned.

13. The method of claim 9, wherein the benefit phase comprises from about 0.05% to about 10%, by weight of the personal cleansing composition, of the lipid bilayer structurant which comprises glyceryl monooleate, glyceryl monostearate, glyceryl monolaurate, or a combination thereof.

14. The method of claim 9, wherein the lipid bilayer structurant comprises glyceryl monooleate.

15. The method of claim 9, wherein the cleansing phase further comprises a cationic polymer.

16. The method of claim 15, wherein the cleansing phase further comprises an associative polymer.

17. The method of claim 16, wherein the cleansing phase further comprises an electrolyte.

18. The method of claim 9, wherein the benefit phase is free of surfactant.

19. The method of claim 9, wherein the benefit phase is anhydrous.

20. A method of improving skin hydration, the method comprising:
providing a personal cleansing composition, comprising:
i. a cleansing phase; and
ii. an anhydrous benefit phase comprising a hydrophobic benefit agent and from about 0.05% to about 5% of a lipid bilayer structurant comprising glyceryl monooleate, glyceryl monostearate, glyceryl monolaurate, or a combination thereof; and
applying the personal cleansing composition to the skin of an individual;
wherein skin hydration improves by about 1.2 Corneometer Units or more, as compared to a water control, at 24 hours after one treatment.
